(19) United States
(12) Patent Application Publication
(10) Pub. No.: US 2016/0220385 A1
(43) Pub. Date: Aug. 4, 2016

(54) MECHANICALLY GUIDED IMPACTOR FOR HIP ARTHROPLASTY

(71) Applicant: ORTHOSOFT INC., Montreal (CA)

(72) Inventors: Bruno FALARDEAU, Verdun (CA); Karine DUVAL, Montreal (CA); Laurence MOREAU-BELANGER, Laval (CA); Francois PARADIS, Boucherville (CA); Di LI, Lasalle (CA); Myriam VALIN, Laval (CA); Benoit PELLETIER, Laval (CA)

(21) Appl. No.: 15/013,402

(22) Filed: Feb. 2, 2016

Related U.S. Application Data
(60) Provisional application No. 62/110,808, filed on Feb. 2, 2015.

(51) Int. Cl. A61F 2/46; A61F 2/34

(52) U.S. Cl. CPC A61F 2/4609; A61F 2/34; A61B 2034/108

(57) ABSTRACT

An impactor for positioning and inserting an acetabular cup into an acetabulum of a pelvis during hip arthroplasty is described. The impactor includes a guide element mounted to an elongated body and including first and second openings aligned with each other to define an axial passage. The first and second openings and the axial passage receive a guide pin therethrough that is pinned in a fixed position to the pelvis. The guide element provides a mechanical orientation guide which restricts an angular orientation of the impactor relative to the guide pin when the guide pin is pinned in the fixed position relative to the pelvis and received through the first and second openings of the guide element. Centering the openings of the guide element relative to the guide pin in the fixed position accordingly achieves a desired orientation of the impactor within a predetermined angular tolerance.

### CROSS-REFERENCE TO RELATED APPLICATIONS

The present application claims priority on U.S. Patent Application No. 62/110,808 filed Feb. 2, 2015, the entire contents of which is incorporated by reference herein.

### TECHNICAL FIELD

The present application relates to computer-assisted surgery using inertial sensors and more particularly to mechanically guided acetabular cup positioning procedure in hip surgery.

### BACKGROUND OF THE ART

In hip arthroplasty, the acetabulum is reamed to subsequently receive therein an acetabular cup. The acetabular cup is an implant that is received within the reamed acetabulum and serves as a receptacle for either a natural femoral head or a femoral head implant. Accordingly, surgical tools such as a reamer and a cup impactor are used in this procedure. One of the challenges in such procedures is to provide an adequate orientation to the acetabular cup. Indeed, an inaccurate orientation may result in a loss of movements, improper gait, and/or premature wear of implant components.

The acetabular cup is typically positioned and inserted into the reamed acetabulum by way of a surgical tool referred to as an impactor. The impactor has a stem at a proximal end of which is mounted the prosthetic acetabular cup. The stem is handled by a user (e.g. surgeon) that impacts the free, distal, end so as to drive the acetabular cup into the acetabulum. It is however important that the user holds the stem of the impactor in a precise three-dimensional orientation so as to ensure that a desired orientation of the acetabular cup is achieved, in terms of inclination and anteversion.

For this purpose, computer-assisted surgery systems are often used to help the user in positioning and orienting the impactor, and therefore the prosthetic acetabular cup mounted thereto, into the desired orientation. Among the various tracking technologies used in computer-assisted surgery, optical navigation and C-arm validation have been used. However, optical navigation requires the use of an associated navigation system, which adds operative time. It also requires pinning an optical reference, visible by the navigation system, on the patient, which adds to the invasiveness of the procedure. Moreover, such optical systems are bound to line-of-sight constraints which can hamper the normal surgical flow. C-arm validation requires the use of bulky equipment and the validation is less cost-effective. Moreover, it does not provide a quantitative assessment of the cup positioning once done, and is generally used post-operatively as opposed to intra-operatively.

Inertial sensors have more recently been used in surgical applications the purposes of determining the orientation of various surgical tools, and are desirable for their cost-effectiveness and the valuable information they provide.

However, there remains a need for an improved instrument used in conjunction with an inertial based computer assisted surgery system, and its associated method of use, which enables the orientation of the impactor , and thus the acetabular cup, to be mechanically guided into its desired orientation.

### SUMMARY

In accordance with one aspect of the present disclosure, there is provided an impactor for positioning and inserting an acetabular cup into an acetabulum of a pelvis during hip arthroplasty, the impactor comprising: an elongated body including a stem having a proximal end and an opposed distal end; a cup-engaging element disposed at the proximal end of the stem, the cup-engaging element being adapted to engage the acetabular cup for insertion into the acetabulum; an impact element disposed at the distal end of the stem and adapted to receive a force used to drive the acetabular cup into the acetabulum, the stem defining a longitudinal axis extending between the cup-engaging element and the impact element, the longitudinal axis thereby defining an impact axis; a guide element mounted to the elongated body, the guide element having first and second openings aligned with each other to define an axial passage extending therebetween, a guide axis centrally disposed within the first and second openings and extending through the axial passage, the first and second openings being spaced apart an axial distance along said guide axis, the first and second openings and the axial passage receiving a guide pin therethrough, the guide pin adapted to be pinned in a fixed position relative to the pelvis, the guide pin defining a pin axis extending longitudinally through a center thereof; and wherein the guide element provides a mechanical orientation guide which restricts an angular orientation of the impactor relative to the guide pin when the guide pin is pinned in the fixed position relative to the pelvis and received through the first and second openings of the guide element, and wherein centering the openings of the guide element relative to the guide pin in the fixed position achieves a desired orientation of the impactor within a predetermined angular tolerance.

There is also provided, in accordance with another aspect of the present disclosure, a patient-specific guide pin installation jig for installing a guide pin in a predetermined fixed position and orientation on a pelvis in preparation for hip arthroplasty, comprising: an acetabular element having an acetabular shell mounted thereto which is configured and formed for a precise mating fit within the acetabulum of the specific patient; a jig body spaced apart from the acetabular element and proximally extending so as to abut with at least one of a rim of the acetabular and another preselected anatomical landmark to define a predetermined position and/or orientation of the jib body; and a pin guide element connected with the jig body and the acetabular element, the pin guide element having a guide hole extending therethrough and adapted to receive at least one of a drill bit and the guide pin, the guide element permitting the guide pin to be pinned to the pelvis in the predetermined position and orientation.

There is also provided a kit for positioning and inserting a prosthetic acetabular cup into an acetabulum of a pelvis during hip arthroplasty, the kit comprising: an impactor as defined immediately above; and a patent-specific guide pin installation jig as defined immediately above.

There is further provided, in accordance with another aspect of the present disclosure, a method for installing an acetabular cup into an acetabulum of a pelvis during hip arthroplasty, comprising: a) seating a guide pin installation jig into the acetabulum; b) using the guide pin installation jig to dispose a guide pin in a pre-planned position and orientation, and driving the guide pin into the pelvis at said pre-planed position and orientation; c) providing an impactor having at least a guide element with first and second axially spaced apart rings circumscribing respective openings which receive the guide pin therethrough, wherein the guide element provides a mechanical orientation guide which restricts an angular displacement of the impactor relative to the guide pin within a predetermined angular tolerance; d) feeding the guide pin through the openings of the guide element of the impactor, and placing a cup-engaging element on a proximal end of the impactor within the acetabulum; e) aligning the impactor at an angular orientation such that the guide pin is substantially centered within the openings of the guide element on the impactor, whereby the impactor is disposed at a pre-planed desired orientation within the predetermined angular tolerance; and f) once the impactor is in the desired orientation as defined by the guide element, impacting the prosthetic acetabular cup into the acetabulum using the impactor.

### BRIEF DESCRIPTION OF THE DRAWINGS

FIG. 1 is a schematic view of a system for navigating instruments in a computer-assisted hip surgery;

FIG. 2 is a perspective view of an impactor in accordance with the present disclosure having a mechanical orientation guide element, for use with the CAS system of FIG. 1;

FIG. 3 is a partial perspective view of an impactor of the present disclosure having the mechanical orientation guide element;

FIG. 4A is a perspective view of the impactor in position for positioning a prosthetic acetabular cup and having an inertial sensor mounted thereto;

FIG. 4B is a block diagram of the inertial sensor of FIG. 4A;

FIG. 5A is a perspective view of a guide pin installation jig, for use in orienting and installing a guide pin used with the impactors of FIGS. 2 to 4B;

FIG. 5B is a perspective view of an alternate guide pin installation jig, for use in orienting and installing a guide pin used with the impactors of FIGS. 2 to 4B;

FIG. 6 is a tracked impactor in accordance with another embodiment, having a guide pin installation jig mounted thereto;

FIG. 7 is a tracked reamer/drill which may be used to create a hole in the pelvis having a predetermined position and orientation for receiving the guide pin therein; and

FIG. 8 is a flow chart of a method for using the impactor in accordance with the present disclosure.

### DETAILED DESCRIPTION

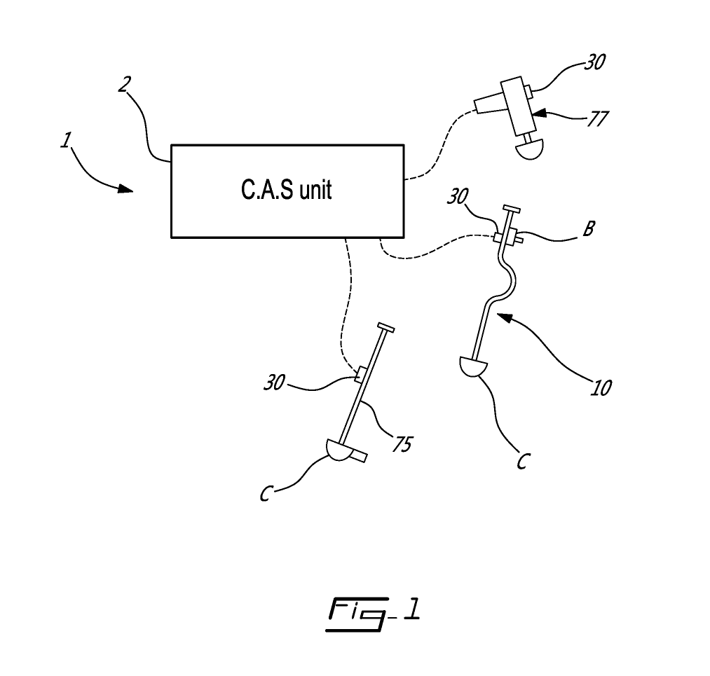

Referring to FIG. 1, a system for navigating surgical instruments in computer-assisted hip surgery is generally shown at 1, and is of the type used to implement the method 300, as will be detailed below. The system 1 comprises generally a computer-assisted surgery (CAS) processing unit 2, shown as a unit in FIG. 1. The CAS processing unit 2 may however be integrated into one or more inertial sensor units 30, also known as “pods”, which comprise “MEMS” (Micro-Electro-Mechanical Sensors) and that are mounted to the various devices and instruments of the system 10. The entire inertial sensor unit 30 may be simply reference to herein as “MEMS” for simplicity. Such MEMS may for example include, but not limited to, accelerometers, gyroscopes and other inertial sensors.

The present surgical tool and method will be generally described herein with respect to use of the device in conjunction with an inertial-based CAS system employing trackable members having inertial-based sensors, such as the MEMS-based system and method for tracking a reference frame as disclosed in United States Patent Application Publication No. US 2011/0218458, and the MEMS-based system and method for planning/guiding alterations to a bone as disclosed in United States Patent Application No. US 2009/0248044, the entire contents of both of which are incorporated herein by reference. While these documents relate more specifically to knee surgery applications wherein the femur and/or the tibia are tracked using such inertial MEMS sensors, it is to be understood that the inertial-based CAS systems and methods described therein can be applied to the tracking of the bone and/or instruments as described herein relating to a hip application.

The inertial sensor units 30 which are mounted to the CAS instruments 5, 7, 10 etc. are in communication with, or incorporate, the processing unit 2 and may thus be equipped with user interfaces to provide the navigation data, whether it be in the form of LED displays, screens, numerical displays, etc. Alternatively, the inertial sensor units A may be connected to a stand-alone CAS processing unit 2 that includes a screen or monitor. The inertial sensor units 30 may comprise the micro-electro-mechanical sensors (MEMS) as described above, and may therefore include one or more of accelerometers, gyroscopes, inclinometers, magnetometers, among other possible inertial sensors.

In one particular embodiment, devices that may be used with the system 1 include an acetabular rim digitizer 75 which is used to define a coordinate system for subsequent navigation, and a surgical instruments/tools such as an impactor 10, an acetabular reamer 77, an impactor guiding pin drill guide, etc.

The CAS processing unit 2 may comprise geometrical data for some of the devices and instruments. Accordingly, when an inertial sensor unit 30 is mounted to one of the devices and instruments, the relation between the device/instrument and a coordinate system of the inertial sensor unit 30 is known. For example, the relation is between an axis or a 3D coordinate system of the device/instrument and the coordinate system of the inertial sensor unit. Moreover, the inertial sensor units 30 may be portable and detachable units, used with one device/instrument, and then transferred to another device/instrument, preserving in the process orientation data of a global coordinate system.

The term “navigation” of instruments is intended to mean tracking at least some of the degrees of freedom of orientation in real-time, or quasi-real time, such that the operator is provided with data calculated by computer assistance (e.g. by the CAS unit 2), which data is representative of hip surgery parameters, such as anteversion and inclination, among other examples. Anteversion may be defined according to an embodiment as the angle between an axis (e.g., impactor axis, cup normal) and the patient frontal plane, the frontal plane being define either by the plane formed by a registration device or a radiographical plane. Anteversion may alternatively be the angle between a medio-lateral axis and a projection of the acetabular axis on the transverse plane (i.e., in which lie the medio-lateral axis and the anterior-posterior axis of the patient). Inclination is the angle between a medio-lateral axis and a projection of the acetabular axis on the frontal plane (i.e., in which lie the medio-lateral axis and the cranial-caudal axis of the patient). The inertial sensors 30 used in the following system, devices and method may be interrelated in a common coordinate system (hereinafter, coordinate system), a.k.a. world coordinate system, global coordinate system, pelvic frame of reference, etc. The common coordinate system serves as a reference to quantify the relative orientation of the different items of the surgery, i.e., the instruments and devices relative to the pelvis.

The instruments of the present disclosure may also be used in conjunction with the systems and methods described in U.S. patent application Ser. No. 14/934,894 filed Nov. 6, 2015 and entitled INSTRUMENT NAVIGATION IN COMPUTER-ASSISTED HIP SURGERY, as well as the systems and methods described in U.S. patent application Ser. No. 14/301,877 filed Jun. 11, 2014, published as US 2014/0364858 and entitled ACETABULAR CUP PROSTHESIS POSITIONING INSTRUMENT AND METHOD, the entire contents of both of which are incorporated herein by reference.

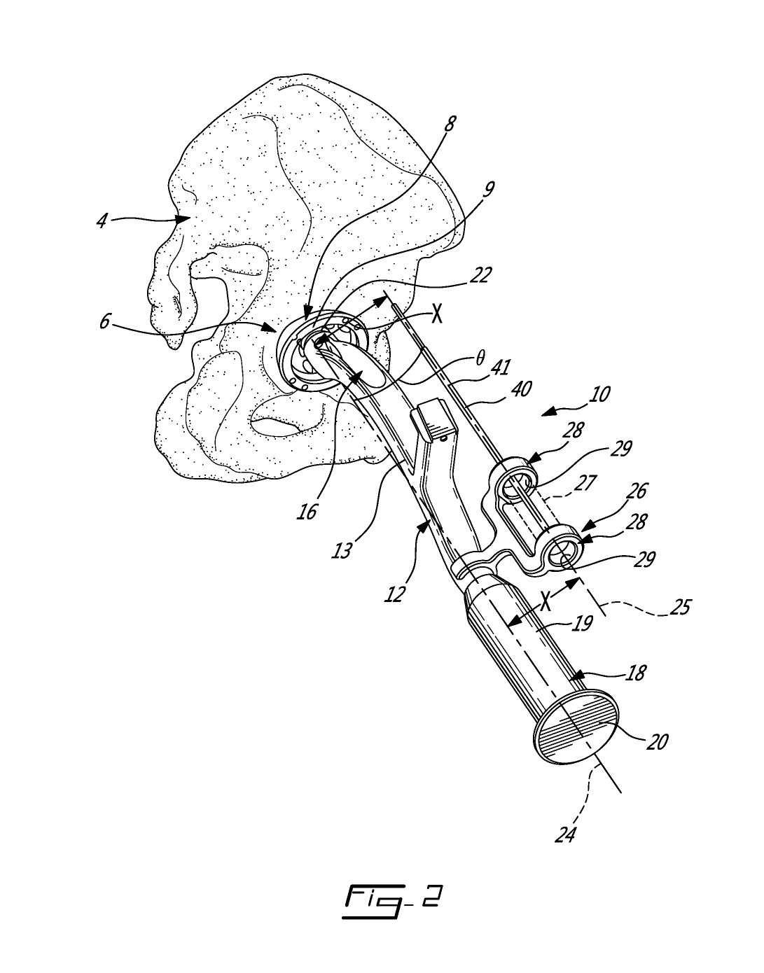

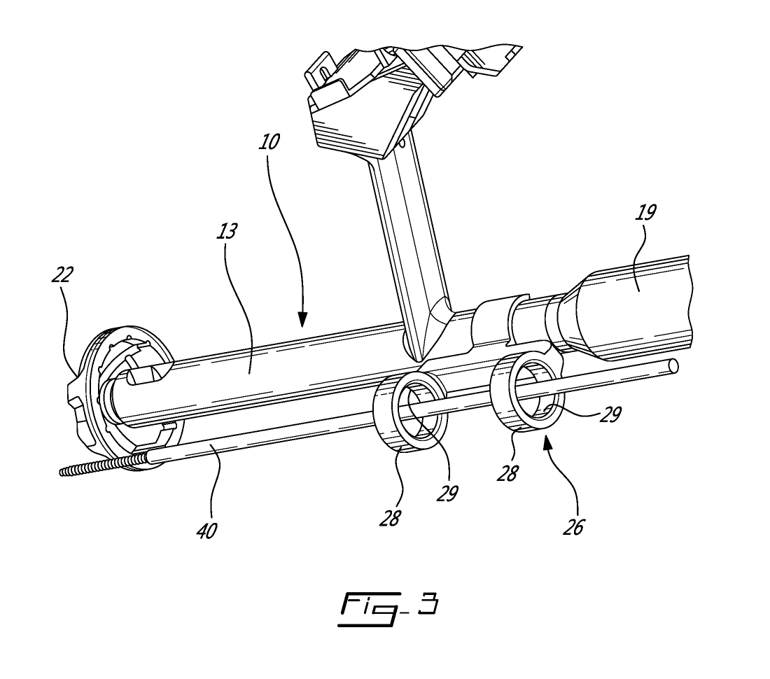

Referring now to FIGS. 2-3, the impactor 10 which may be used with the CAS processing unit 2 of the above-described CAS system will now be described in further detail. The impactor 10 of the present disclosure is used for positioning and inserting a prosthetic acetabular cup 8 into an acetabulum 6 of a pelvis 4. Typically, during hip arthroplasty the acetabulum is first reamed by a reamer tool, and then subsequently receives a prosthetic acetabular cup therein. The impactor 10 is accordingly used to accurately and repeatably position and orient the prosthetic acetabular cup, and then insert the acetabular cup 8 in place within the acetabulum 6 of the pelvis 4.

The impactor 10 includes generally a body 12 including an elongated arm or stem 13 having a proximal end 16 and an opposed distal end 18. The stem 13 may be either straight or curved. The distal end 18 of the stem 13 includes a handle 19 terminating in an impact element 20 (such as an impact anvil) adapted to receive an impact force used to drive the acetabular cup 8 into the acetabulum 6.

A head or cup-engaging element 22 is disposed at the proximal end 16 of the stem 13, the cup-engaging element 22 being adapted to have the prosthetic acetabular cup 8 mounted thereto, such that the acetabular cup 8 can be positioned as required by the operator of the impactor 10 (e.g. a surgeon) using the handle 19 and then inserted into the acetabulum 6 by applying a force (e.g. an impact force) on the impact element 20 to drive the acetabular cup 8 into the reamed acetabulum 6.

A longitudinal axis 24 extends through the body 12 of the impactor 10, although does not necessary extend through the center of the stem 13 given that it may be curved (as shown in FIG. 2). More specifically, the longitudinal axis 24 extends longitudinally between the cup-engaging element 22 and the impact element 20 such as to define an impact axis. The longitudinal axis 24 is also aligned with a cup axis of the acetabular cup 8, such that impact forces applied to the impact element 20 are transmitted through the impactor 10 along the longitudinal axis 24 thereof and along the cup axis of the prosthetic acetabular cup 8. The head or cup-engaging element 22 may therefore be arranged such that the longitudinal axis 24 of the impactor 10 is normal to a plane in which lies the rim 9 of the acetabular cup 8. Stated differently, in one embodiment, the axis 24 of the impactor body 12 is coincident with the axis of the cup 8, which cup axis is the reference to orient the cup in the acetabulum.

Referring still to FIGS. 2-3, the impactor 10 of the present disclosure further includes a guide element 26 that is mounted to the stem 13 of the impactor body 12 and that protrudes from the stem 13 in a direction that is transverse (though not necessarily perpendicular) relative to the longitudinal impact axis 24. The guide element 26 may be either integrally formed with the remainder of the stem 13 forming the elongated body 12 of the impactor 10, or alternately may be separately formed and removably attached thereto. In the embodiment shown in FIG. 2, the guide element 26 is fastened in place on the stem 13 of the impactor body 12, in a predetermined position and orientation thereon. The guide element 26 fastened in this manner may be either removably fastened, for example using a quick-connect type snap engagement, or may be more permanently fastened using suitable fasteners or welds, etc.

The guide element 26 of the impactor 10 provides a mechanical orientation guide which restricts an angular orientation of the impactor relative to a fixed guide pin 40 that is fixed in place to the pelvis in a manner that will be described in further detail below. The guide pin 40 may define a pin axis 41 extending longitudinally through a center thereof.

More particularly, the guide element 26 includes at least first and second axially spaced apart rings 28, which each circumscribe an opening 29. The two axially spaced apart openings 29 are substantially aligned such as to define an axial passage 27 extending therebetween, and through which the guide pin 40 passes. This axial passage 27 may be only partially enclosed (i.e. by the rings 28), as sown in the embodiment of FIG. 2, or may alternately be fully enclosed (i.e. the axially spaced apart rings 28 may in fact form opposed ends of a fully circumferentially enclosed cylinder). In the case of the later, i.e. the fully enclosed cylinder which defines the axial passage 27 therethrough, the openings 29 are nevertheless defined at each of the opposed open ends of the cylinder, through which the pin 40 passes. Regardless, by centering the axially spaced apart openings 29 of the guide element 26 relative to the fixed guide pin 40, a desired orientation of the impactor can be easily achieved within a predetermined angular tolerance, and can be rapidly and accurately visually confirmed by the surgeon (for example, by ensuring that the pin 40 is centered within both of the openings 29 of the guide element 26).

The centers of the two openings 29 of the guide element 26 therefore are disposed along a guide axis 25 that extends concentrically through both axially spaced openings 29. The guide axis 25 is parallel to the longitudinal axis 24 of the impactor 10 and transversely spaced apart therefrom a predetermined transverse distance X. This transverse distance X is selected to be the same as the known distance between the axis of the impactor 24 (i.e. extending through the center of the acetabulum) and the pin axis 41 of the guide pin 40. The diameters of these openings 29 are selected such that an angular range is defined for the acetabular cup 8 (such as ±10 degrees, for example, from the optimal orientation of the acetabular cup as defined by the pin axis 41).

In one particular embodiment of the impactor 10, the circular openings 29 defined by the rings 28 of the guide element 26 have a diameter of about 20 mm, and these openings 29 are positioned about 70 mm apart (i.e. the axial distance between the most proximal opening 29 in the proximal ring 28 and the most distal opening in the distal ring 28). In terms of axial positioning of the guide element 26, the most proximal opening 29 of the proximal ring 28 is positioned about 220 mm from the pelvis 4 and therefore from the base of the guide pin 40. The guide pin 40 employed in this particular embodiment has a diameter of about 4 mm. Accordingly, the orientation θ of the longitudinal axis 24 of the impactor 10 to be limited to within ±2.6 degrees relative to the pin axis 41 of the guide pin 40, which is obtained by calculating: θ=Tan⁻¹(10/220). This therefore results in a total possible angular tolerance range of 5.2 degrees.

With the guide pin 40 in place within the pelvis 4, the impactor 10 can accordingly be axially displaced toward and away from the bone, with the guide pin 40 remaining within the openings 29 of the guide element 26. As such, the guide element 26 is used to orient the impactor at a desired angular orientation, as defined by the angular orientation of the guide pin 40, an allows for a predetermined amount of angular tolerance (error)—such as the ±2.6 degrees in the example above—while still providing mechanical limits, by way of the rim defining each opening 29 against which the pin abuts to form a mechanical stop or limiter, to the maximum angular deviation away from the desired orientation within the predetermined angular tolerance.

The amount of angular tolerance, and thus the allowable maximum angular deviation of the orientation of the impactor, can be selected and/or modified as required by varying one or more of a number of parameters, including: the axial position of the guide element 26 along the body of the impactor; the size of the openings 29 of the guide element 26; the axial spacing between each of the two openings 29 of the guide element; and the diameter of the guide pin 40.

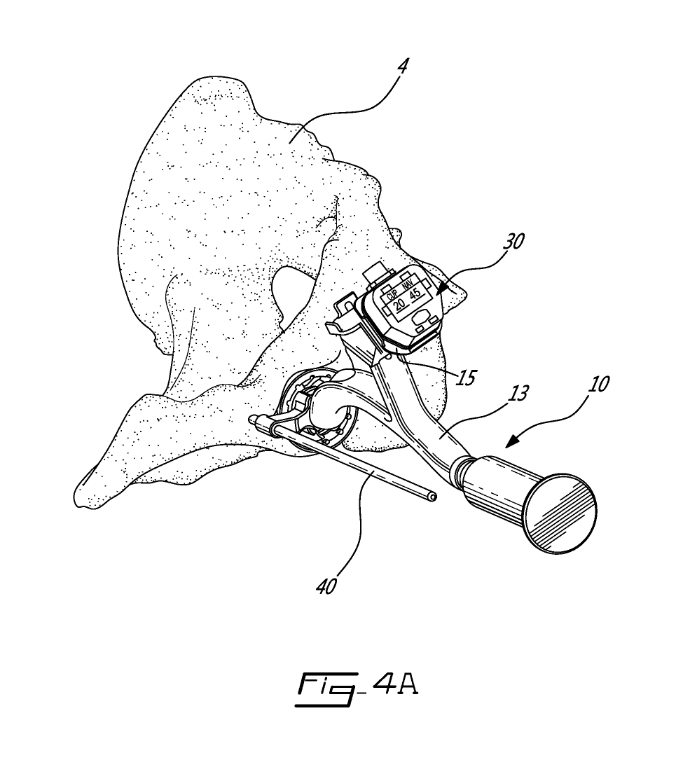

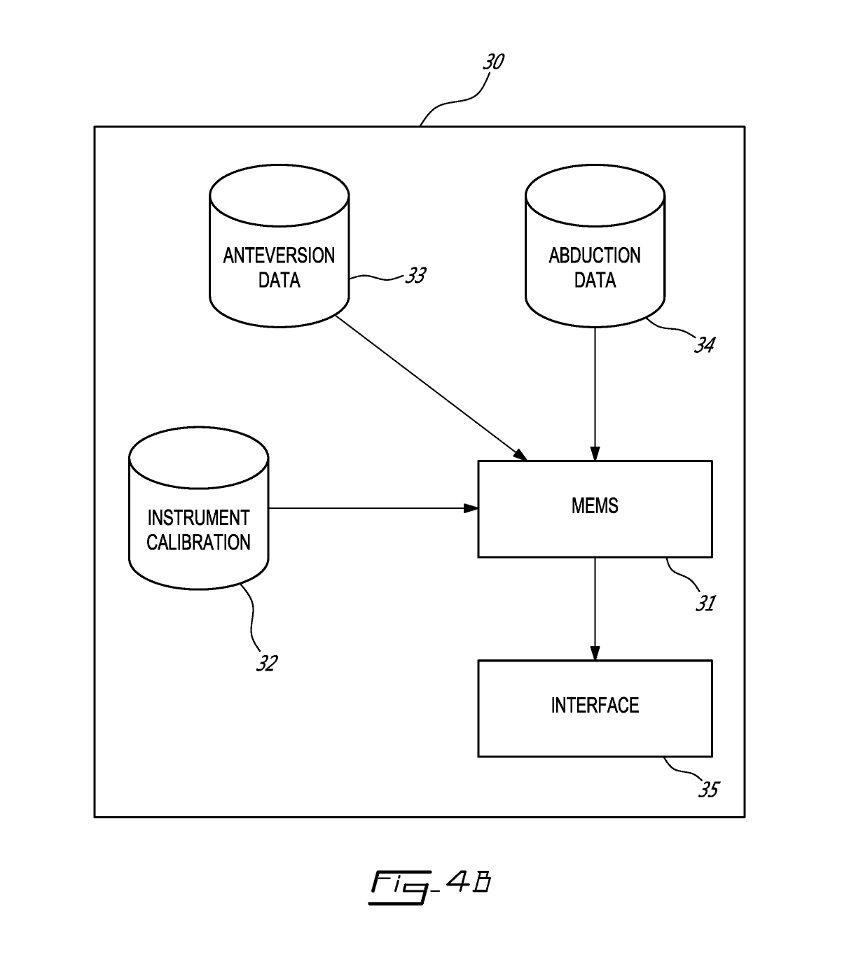

Referring now to FIG. 4A, the impactor 10 may further have at least one of the above-mentioned inertial sensor units 30 mounted to pod-receiving base 15 located on the stem 13 or elsewhere on the body 12 of the impactor 10. The exact location of the pod-receiving base 15, and thus the inertial sensor unit 30 removably mounted thereto, is disposed in a known position and orientation relative to the longitudinal axis 24 of the impactor 10, such as to track at least the orientation of the impactor 10. The impactor 10 shown in FIG. 4A is a described above, however it is depicted with the guide element 26 removed.

The inertial sensor unit 30 is as described above, and is shown in greater detail in FIG. 4B. The inertial sensor unit 30 comprises appropriate micro-electromechanical sensor(s) 31 (e.g., accelerometers, gyroscopes, inclinometers, or the like) and associated electronics and processor chosen to perform the tasks described hereinafter by outputting real-time orientation data related to the movements of the inertial sensor unit 30. The inertial sensor unit 30 is preprogrammed as a function of the pre-operative planning to perform the tasks described hereinafter. It is however known that the inertial sensor unit 30 must be calibrated for its readings to be related to the orientation of the pelvis, and may have a patient-specific file for calibration and navigation. As a starting point, instrument calibration data 32 is for instance provided for the inertial sensor unit 30 to be aligned at initialization with the longitudinal axis 24 of the instrument 10. The instrument calibration data is based on a planned geometric relation between an initial reference orientation of the instrument 10 and an anatomical landmark(s) of the pelvis, the calibration data being used to calibrate the inertial sensor unit 30 relative to the pelvis for the inertial sensor unit 30 to be able to produce the orientation output based on the preoperative planning. The patient-specific file may also include a desired acetabular cup orientation data based on preoperative planning. The desired acetabular cup orientation data may for instance consists of anteversion angle data 33 and/or abduction angle data 34 also programmed into the inertial sensor unit 30, as a function of the pre-operative planning, the anteversion angle data 33 being representative of the anteversion angle at which the operator wants the cup to be, while the abduction angle data 34 is representative of the abduction angle at which the operator wants the cup to be. An interface 35, of any appropriate form, will also be provided as part of the inertial sensor unit 20, directly thereon or remotely therefrom. The interface 35 may be in the form of LEDs signaling a proper/improper orientation, or being a screen giving the numeric angle values.

When maintaining the implant cup in the acetabulum, prior to impacting, the instrument 10 is arranged to be vertical (i.e., an initial reference orientation). According to an embodiment, the inertial sensor unit 30 is used to guide the operator in achieving verticality of the instrument 10. For instance, LEDs may be provided on inertial sensor unit 30 to provide visual indication when appropriate verticality is reached.

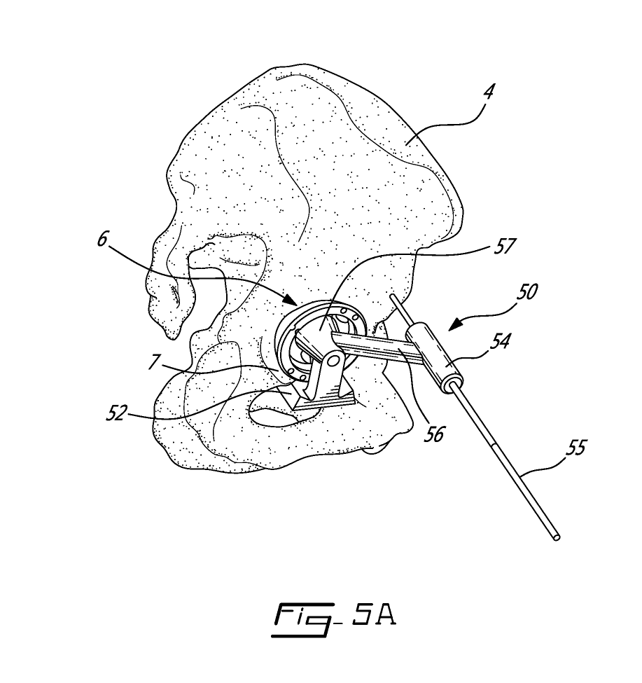

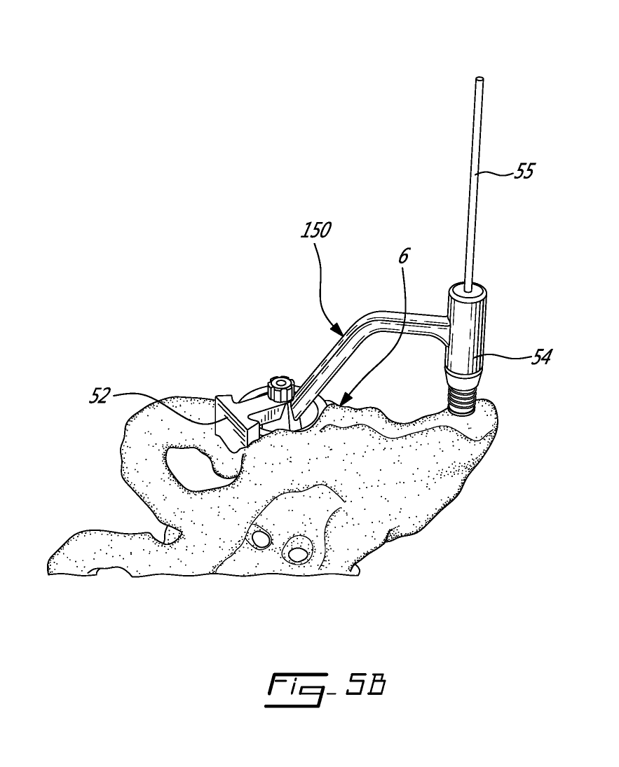

Referring now to FIGS. 5A-5B, guide pin installation jigs 50 and 150 may be used to accurately position and orient the guide pin 40 relative to the acetabulum 6 of the pelvis 4. In one possible embodiment, the guide pin installation jig 50,150 is a patient-specific instrument (PSI), which is specifically configured and formed to adapt to a given patient's acetabulum 6 once it has been reamed in preparation for receiving the prosthetic acetabular cup 8.

The PSI jigs 50,150 include an acetabular element 57 which is at least partially received within the acetabulum 6 of the pelvis 4. The acetabular element 57 may be attached to an acetabular shell (i.e. not the final prosthetic cup that will actually be implanted) having a size and shape specifically configured to fit within the acetabulum 6 of the specific patent's pelvis 4. This may be either a provisional acetabular shell that is sized to fit within the non-reamed acetabular, or alternately one which is sized and configured to fit within the acetabulum after it has been reamed. The PSI jig 50,150 may also includes a jig body 52 which mates with either the rim 7 of the acetabulum 6 or another preselected anatomical landmark which allows the guide pin to be oriented in a desired orientation which is planned pre-operatively. Because the jig 50,150 is, in this embodiment, a PSI jig, it is produced such as to precisely position and orient the hole in the pelvis 4, which will receive the guide pin 40, relative to the patient's acetabulum 6. The PSI jig 50,150 therefore also includes a drill guide element 54 having a drill guide hole extending therethrough, which is used to guide a drill bit 55 that is used to drill the hole in the pelvis at the pre-planned orientation as defined by the drill guide element 54 of the PSI jig 50,150. Alternately, the guide pin 40 can simply be driven directly into the bone using the drill guide 54.

In one embodiment, the guide pin installation jig 50 may include an adjustable arm 56, the arm 56 being adjustable in length and/or orientation per-operatively based on output of pre-operative planning data (such as CT-scan, 2 x-rays, etc.). Using pre-operative planning, the optimal orientation of the acetabular cup is first determined, and from this the orientation of the drill guide 54, which shall be parallel to the optimal orientation of the acetabular shell axis, is determined based on the pelvic coordinate system as defined using any standard definitions (e.g. Lewinneck pelvic coordinate system). Once the arm 56 of the guide pin installation jig 50 is in position, the drill guide 54 is used to fix the guide pin 40 on the pelvis bone 4 in the predetermined orientation.

The jigs 50,150 are but one possible guide pin positioner which can be used to dispose the guide pin 40 in the predetermined (pre-planned) position and orientation relative to the acetabulum. For example, the guide pin positioner may form part of a separate acetabulum digitizer which mates with the acetabulum.

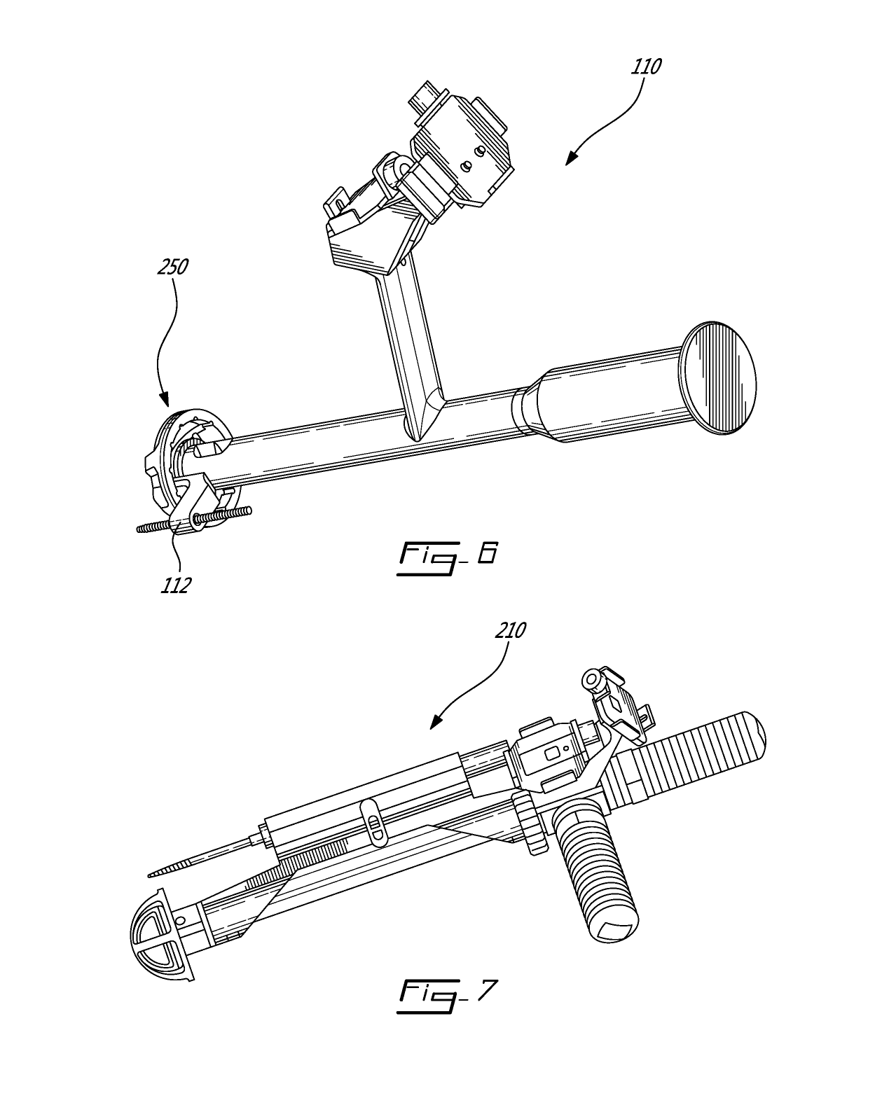

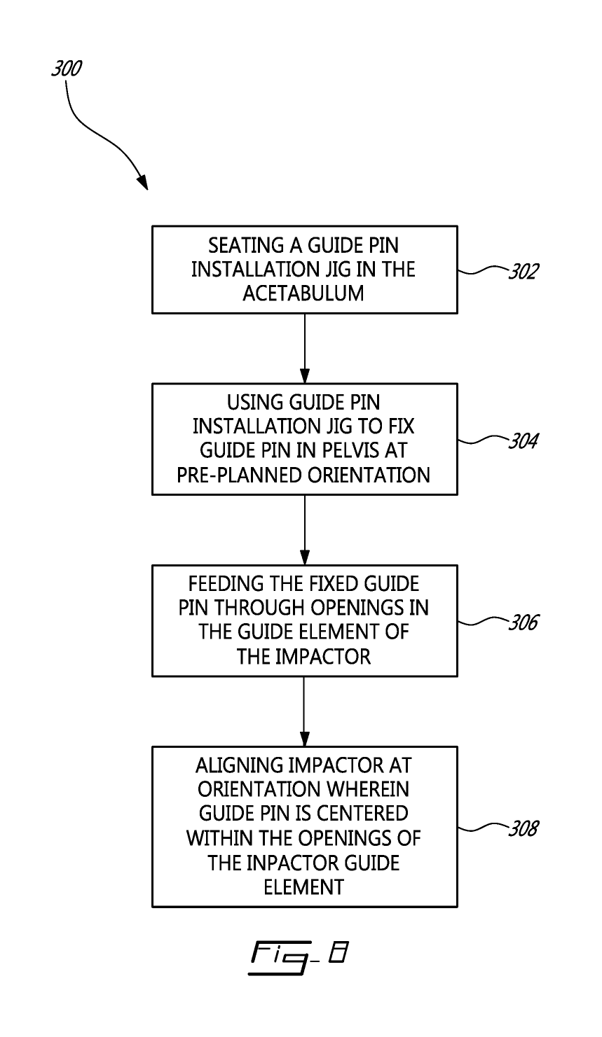

Alternately, the guide pin positioner may include the alternate embodiments, such as the tracked impactor 110 of FIG. 6, having a guide pin installation jig 250 including a drill/pin guide element 112, or the tracked reamer/drill 210 as depicted in FIG. 7. Using the guide pin installation jig 250 of FIG. 6 provides the added advantage that the surgeon can place the guide pin 40 at any desired position on the pelvis, and the orientation of the pin is guided and set by the navigation of the tracked impactor 110 to which at least one MEMS pod 30 is mounted. This enables the surgeon to select a desired location around the acetabulum where the drill hole for the guide pin is to be positioned. Further, by using an adjustable drill guide 112, or alternately having different sizes of drill guides 112 which can be positioned in place on the installation jig 250, a distance between the axis of the pin 40 and the eventual impactor axis within the acetabulum can therefore be selected as required by the surgeon. Once this distance is selected, the same distance is then used by the surgeon for the guide element 26 of the impactor 10.

Referring now to FIG. 8, the method 300 of installing an acetabular cup using the impactor 10 as described herein generally comprises: step 302, which includes, prior to or following reaming of the acetabulum, seating a guide pin installation jig into the acetabulum; step 304, which includes using the guide pin installation jig to drive a guide pin into the pelvis at a pre-planned orientation; step 306, which includes removing the guide pin installation jig and placing the impactor 10 in position, with the guide pin 40 extending through the openings 29 of the guide element 26 mounted to the impactor 10; step 308, which includes aligning the impactor at an angular orientation such that the guide pin 40 is substantially centered within both openings 29 of the guide element 26 on the impactor; and 5) once the impactor is in the desired orientation based on the mechanical guidance of the guide element 26, impacting the prosthetic acetabular cup 8 into the acetabulum using the impactor 10.

While the methods and systems described herein have been described and shown with reference to particular steps performed in a particular order, it will be understood that these steps may be combined, subdivided or reordered to form an equivalent method without departing from the teachings of the present invention. Accordingly, the order and grouping of the steps is not a limitation of the present invention.

1. An impactor for positioning and inserting an acetabular cup into an acetabulum of a pelvis during hip arthroplasty, the impactor comprising:
an elongated body including a stem having a proximal end and an opposed distal end;
a cup-engaging element disposed at the proximal end of the stem, the cup-engaging element being adapted to engage the acetabular cup for insertion into the acetabulum;
an impact element disposed at the distal end of the stem and adapted to receive a force used to drive the acetabular cup into the acetabulum, the stem defining a longitudinal axis extending between the cup-engaging element and the impact element, the longitudinal axis thereby defining an impact axis;
a guide element mounted to the elongated body, the guide element having first and second openings aligned with each other to define an axial passage extending therebetween, a guide axis centrally disposed within the first and second openings and extending through the axial passage, the first and second openings being spaced apart an axial distance along said guide axis, the first and second openings and the axial passage receiving a guide pin therethrough, the guide pin adapted to be pinned in a fixed position relative to the pelvis, the guide pin defining a pin axis extending longitudinally through a center thereof; and
wherein the guide element provides a mechanical orientation guide which restricts an angular orientation of the impactor relative to the guide pin when the guide pin is pinned in the fixed position relative to the pelvis and received through the first and second openings of the guide element, and wherein centering the openings of the guide element relative to the guide pin in the fixed position achieves a desired orientation of the impactor within a predetermined angular tolerance.

2. The impactor as defined in claim 1, where outer rims of the first and second openings provide a physical stop which defines a maximum angular deviation within the predetermined angular tolerance of the impactor relative to the fixed guide pin.

3. The impactor as defined in claim 1, wherein the guide element includes first and second rings respectively circumscribing the first and second openings, the first and second openings being circular, and the first and second rings being axially spaced apart said distance relative to the longitudinal axis.

4. The impactor as defined in claim 3, wherein at least the first and second rings of the guide element protrude from the stem of the elongated body transversely relative to the longitudinal axis.

5. The impactor as defined in claim 3, wherein the axial passage is only partially enclosed by the first and second rings, the first and second rings defining a gap therebetween within the axial passage.

6. The impactor as defined in claim 1, wherein the guide element includes an tubular cylinder defining the first and second openings at opposed ends thereof, the axial passage extending through the tubular cylinder between the first and second openings located at opposed ends of the tubular cylinder.

7. The impactor as defined in claim 1, wherein the guide element is removably attached to the stem of the elongated body in a fixed and predetermined position and orientation relative thereto.

8. The impactor as defined in claim 1, wherein the first and second openings of the guide element are configured to permit the predetermined angular tolerance to be at most ±10 degrees between the longitudinal axis of the stem and the pin axis of the guide pin.

9. The impactor as defined in claim 8, wherein the first and second openings of the guide element are configured to permit the predetermined angular tolerance to be about ±2.6 degrees between the longitudinal axis of the stem and the pin axis of the guide pin.

10. The impactor as defined in claim 9, wherein the first and second openings have a diameter of about 20 mm, the axial distance separating the first and second openings being about 70 mm, and a most proximal one of the first and second openings being located about 220 mm from the up-engaging element disposed at the proximal end of the stem.

11. The impactor as defined in claim 1, wherein the guide axis of the guide element is substantially parallel to the longitudinal axis of the stem of the elongated body.

12. The impactor as defined in claim 11, wherein the guide axis and the longitudinal axis are spaced apart by a predetermined transverse distance.

13. The impactor as defined in claim 1, further comprising at least one inertial sensor unit mounted to the elongated body, the inertial sensor being operable to determine and output real-time data representative of at least an orientation of the impactor in space.

14. The impactor as defined in claim 13, wherein the inertial sensor unit includes a micro-electro-mechanical sensor (MEMS) having one or more accelerometer, gyroscope, inclinometer and/or magnetometer.

15. The impactor as defined in claim 14, wherein the MEMS includes one or more user interfaces integrated therewith.

16. The impactor as defined in claim 15, wherein the user interfaces of the MEMS include at least one of an LED display, a screen and a numerical display.

17. The impactor as defined in claim 16, wherein the MEMS includes an LED display, the LED display signaling a proper and/or improper orientation of the impactor.

18. The impactor as defined in claim 16, wherein the MEMS includes a screen operable to depict numerical angle values of the orientation of the impactor.

19. The impactor as defined in claim 13, wherein the inertial sensor unit is in wireless communication with a computer-assisted surgery (CAS) processing unit of a (CAS) system remote from the impactor.

20. A patient-specific guide pin installation jig for installing a guide pin in a predetermined fixed position and orientation on a pelvis in preparation for hip arthroplasty, comprising:
an acetabular element having an acetabular shell mounted thereto which is configured and formed for a precise mating fit within the acetabulum of the specific patient;
a jig body spaced apart from the acetabular element and proximally extending so as to abut with at least one of a rim of the acetabular and another preselected anatomical landmark to define a predetermined position and/or orientation of the jib body; and
a pin guide element connected with the jig body and the acetabular element, the pin guide element having a guide hole extending therethrough and adapted to receive at least one of a drill bit and the guide pin, the guide element permitting the guide pin to be pinned to the pelvis in the predetermined position and orientation.

21. A kit for positioning and inserting a prosthetic acetabular cup into an acetabulum of a pelvis during hip arthroplasty, the kit comprising:
an impactor as defined in claim 1; and
a patent-specific guide pin installation jig, the patent-specific guide pin installation jig comprising:
an acetabular element having an acetabular shell mounted thereto which is configured and formed for a precise mating fit within the acetabulum of the specific patient;
a jig body spaced apart from the acetabular element and proximally extending so as to abut with at least one of a rim of the acetabular and another preselected anatomical landmark to define a predetermined position and/or orientation of the jib body; and
a pin guide element connected with the jig body and the acetabular element, the pin guide element having a guide hole extending therethrough and adapted to receive at least one of a drill bit and the guide pin, the guide element permitting the guide pin to be pinned to the pelvis in the predetermined position and orientation.

22. A method for installing an acetabular cup into an acetabulum of a pelvis during hip arthroplasty, comprising:
a) seating a guide pin installation jig into the acetabulum;
b) using the guide pin installation jig to dispose a guide pin in a pre-planned position and orientation, and driving the guide pin into the pelvis at said pre-planed position and orientation;
c) providing an impactor having at least a guide element with first and second axially spaced apart rings circumscribing respective openings which receive the guide pin therethrough, wherein the guide element provides a mechanical orientation guide which restricts an angular displacement of the impactor relative to the guide pin within a predetermined angular tolerance;
d) feeding the guide pin through the openings of the guide element of the impactor, and placing a cup-engaging element on a proximal end of the impactor within the acetabulum;
e) aligning the impactor at an angular orientation such that the guide pin is substantially centered within the openings of the guide element on the impactor, whereby the impactor is disposed at a pre-planed desired orientation within the predetermined angular tolerance; and
f) once the impactor is in the desired orientation as defined by the guide element, impacting the prosthetic acetabular cup into the acetabulum using the impactor.
